(19) United States
(12) Patent Application Publication
(10) Pub. No.: US 2007/0167778 A1
(43) Pub. Date: Jul. 19, 2007

(54) Acoustic interrogation system and method of operation

(76) Inventor: Robert J. Crowley, Sudbury, MA (US)

Correspondence Address: Donald N. Halgren, 35 Central Street, Manchester, MA, 01944, US

(21) Appl. No.: 11/337,814

(22) Filed: Jan. 23, 2006

Related U.S. Application Data
(60) Provisional application No. 60/753,771, filed on Dec. 23, 2005.
(60) Provisional application No. 60/754,076, filed on Dec. 27, 2005.

(51) Int. Cl. A61B 8/00

(52) U.S. Cl. 600/443

(57) ABSTRACT

A system for insonification and data collection of biological specimens and samples. The system comprises one or more transducers held in proximity to a cell or group of cells for a period of time and in a specific relationship. Methods of generating acoustic signals that are sent and received to and from the transducers and the cells over specific time periods and recorded data is compared over time periods from several hours to a several day period are described. The device features an operable system comprising a multiplicity of transducer elements in close proximity to and acoustically coupled with standardized polymer array plates that are rapidly interchangeable.

### BACKGROUND OF THE INVENTION

1. Field of the Invention

The present invention relates to biological tissue sampling arrangements, and more particularly to acoustic tissue-sampling systems over an extended period of time, and is based upon U.S. Provisional Patent Applications 60/753,771 filed Dec. 23, 2005, and 60/______, filed Dec. 27, 2005, each of which are incorporated herein by reference in their entirety.

2. Background of the Art

Tissue characterizations systems employing spectral analysis of ultrasonic signals are generally designed for the detection and location of tissue types, density and morphology, either in-vitro or in-vivo. Such systems may work in either the through transmission mode, or, more commonly, in the pulse-echo mode, where an acoustic pulse transmitted from one or more transducers is detected after being reflected by the tissue undergoing investigation.

In the case of the pulse-echo type system, a typical signal processing method consists of capturing a returning signal and performing spectral analysis to that signal. Either a single pulse or a train of pulses directed at a region of interest may be processed in this manner. Various methods may be used to correlate the acoustic spectrum with suspected or known forms of tissue, or of diseases, such as cancer, or atherosclerosis. These tissue characterization systems have been used experimentally to analyze known disease states and also commercially as an adjunct to the visualization of cancerous tissue of the breast and other organs of the body, such as the prostate. The systems are useful but have certain drawbacks. One major drawback is the need to transmit through intervening tissue which transmission attenuates as a function of frequency and depth and varies from patient to patient, and also is subject to operator variability. Another limitation is the short time period generally used to collect the spectral information, which may change over the course of hours or days, the changes which may indicate important trends in cellular states within diseases for example, or the rate and spread of a disease, or a change over time as a result of the application of a drug, an external energy source, a molecule, or a therapy.

It would be desirable if such a system could overcome these limitations and have the consistency and longer-term use characteristics needed for interrogation of cells over time corresponding to cellular division, cellular aging and cell death, and be simple, practical, and adaptable to a wide variety of laboratory apparatus and medical device configurations, and be repeatable, practical and inexpensive.

It is an object of the present invention to provide a set of components including at least one acoustic transducer that is held in proximity to at least one cell in a consistent and repeatable manner, and to detect an acoustic change in that cell over an extended period of time from between about 2 hours to greater than about 48 hours.

It is a further object of the invention to perform interrogation of at least one human cell over an extended period of time in an in-vitro, controlled environment. It is a further object of the invention to establish a high throughput method for the interrogation of multiple samples of at least one human cell during prolonged periods, and in a consistent, repeatable manner, and that yields a spectral pattern that may be characterized over a time frame from about 2 hours to greater than about 48 hours.

It is the object of the invention to apply the principles of consistency and repeatability that are defined by the method to implement a tissue characterization platform adaptable to medical devices, such as catheters, trocars, needles, free-floating bodies that may be conveyed through the vascular system, the gut, the lymphatic system, and to implantable devices, such as stents, filters, artificial joints, heart valves, and the like.

In one embodiment, a transducer array system is used in a through transmission mode in conjunction with standardized cell culture apparatus such as Petri dishes and multi-well arrays made of polymeric materials that are acoustically transmissive.

In another embodiment, a pulse-echo transducer insonifies and collects acoustic spectral data from at least one cell in a chamber, wherein the pulse echo transducer is arranged to transmit through at least one wall of the chamber. In one aspect, the chamber is made of an acoustically transmissive material such as styrene. In another aspect the chamber is a sterile, non-pyrogenic multi-well plate with a low evaporation lid. In one embodiment, the chamber and transducers are held in a fixed relationship to each other and placed in a controlled environment such as an incubator, and signal wires are connected to an array of transducers arranged to correspond to the locations and positions of the chambers in a multi-well plate.

In another embodiment, the invention consists of a small transducer arranged in fixed relationship to an implantable device that is read over a prolonged period of time from about 2 hours to greater than about 48 hours. In one aspect, the small transducer is arranged to conform to the size of at least one cell.

### BRIEF SUMMARY OF THE INVENTION

The present invention comprises a biological sample acoustic interrogation system having at least one acoustic transducer for interrogating that biological sample. The system comprises an acoustic transducer arrangement positioned relative to an acoustically transmissive support, which support is arranged in acoustically transmissive contact with the biological sample. The acoustic transducer arrangement is arranged in timed acoustic interrogating contact with the support. The timed acoustic interrogating contact comprises a period of about 2 hours to greater than about 48 hours. The acoustic transducer arrangement may comprise a plurality of individual transducers. The acoustic transducer arrangement is preferably fixedly arranged with respect to the acoustically transmissive support. The acoustic transducer arrangement may be movably arranged with respect to the acoustically transmissive support. The acoustically transmissive support may be movable arranged with respect to the acoustic transducer arrangement. The biological sample preferably comprises at least one biological cell. The acoustically transmissive material may be a polymeric chamber. The polymeric chamber may be comprised of a multi-well plate.

The invention also comprises a method of acoustically interrogating biological samples over an extended time period comprising one or more of the following steps of: arranging a biological sample for interrogation; placing the sample in acoustical communication with an acoustic transducer arrangement; energizing the transducer so as to provide a return signal during an initial time-interrogation event; energizing the transducer again so as to provide a further return signal during a later time-interrogation event; and characterizing differences in spectral patterns generated during the initial time-interrogation event and the later time-interrogation event so as to determine sample type and morphology. The initial time-interrogation event and the later time-interrogation event may be separated by at least 48 hours. The acoustic transducer arrangement may comprise a pair of spaced-apart acoustic transducers. The spaced apart transducers may have the sample arranged therebetween. The transducer arrangement may be disposed within an implantable medical device. The medical device may be a stent. The medical device may be a tissue filter. The medical device may be an artificial joint. The medical device may be a heart valve. The acoustic transducer arrangement may be comprised of a pulse-echo transducer. The acoustic transducer arrangement may be comprised of a through transmission transducer. The transducer arrangement may be sized to correspond dimensionally to a sample size. The method may including: placing the sample in an acoustically transmissive chamber. The acoustically transmissive chamber may be comprised of styrene. The chamber may be comprises of a sterile, non-pyrogenic multiwell plate. The chamber may have a low evaporation lid arranged thereon. The chamber and the transducer arrangement may be disposed in a fixed relationship with one another. The method may include: placing the chamber in a controlled environment prior to energizing of the transducer. The method may include: placing the transducer arrangement in an implantable device and placing the implantable device in a living body part.

The invention also comprises a through-transmission acoustic system for the repeatable, consistent interrogation of a biological sample of tissue over a period of time, comprising: at least two transducers arranged in a circuit controlled by a central processing unit; an oscillator arranged in the circuit, to generate a pulse of specific electrical waveforms; an amplifier arranged in the circuit to increase the strength of the pulse of one of the transducers into acoustic energy; an arrangement of electrodes attached to the transducer arrangement, to conduct the pulse into an acoustic wave directed to the sample; a second of the transducers arranged to receive the acoustic wave from the one of the transducers and convert the wave into an electrical waveform; a second amplifier in the circuit arranged to condition the wave into time and frequency domains; and a processor for display of spectral components of a signal received by the second transducer over a period of time to present quantitative information relative to the sample changes over the time period of interrogation. The time period of interrogation of the biological sample of tissue preferably comprises a range of at least two hours up to at least about 48 hours. The two transducers are preferably disposed in a tissue sample-contacting chamber. The tissue sample-contacting chamber may be arranged in-vivo. The chamber may be an in-vivo medical device. The medical device may consist of a stent. The medical device may consist of a catheter. The medical device may consist of a filter. The medical device may consist of a trocar. The medical device may consist of an artificial joint. The medical device may consist of a needle. The medical device may consist of a free-floating body conveyable through a vascular system. The medical device may consist of a heart valve.

The invention also comprises a pulse-echo acoustic system for the repeatable, consistent interrogation of a biological sample of tissue over a period of time, comprising: a transducer arrangement disposed in a circuit controlled by a central processing unit; an oscillator arranged in the circuit, to generate a pulse of specific electrical waveforms; an amplifier arranged in the circuit to increase the strength of the pulse of one of the transducers into acoustic energy; an electrode attached to the transducer arrangement, to conduct the pulse into an acoustic wave directed to the sample; a switch arranged to control and re-direct an echo of the acoustic wave from the transducer arrangement and convert the echo into an electrical waveform; a receive amplifier in the circuit arranged to condition the wave into time and frequency domains; and a processor for display of spectral components of a signal redirected by the switch over a period of time to present quantitative information relative to the sample changes over the time period of interrogation. The time period of interrogation of the biological sample of tissue comprises a range of at least two hours up to at least about 48 hours. The transducer arrangement is preferably disposed in a tissue sample-contacting chamber. The tissue sample-contacting chamber may be arranged in vivo. The chamber may be an in-vivo medical device. The medical device may consist of a stent. The medical device may consist of a catheter. The medical device may consist of a filter. The medical device may consist of a trocar. The medical device may consist of an artificial joint. The medical device may consist of a needle. The medical device may consist of a free-floating body conveyable through a vascular system. The medical device may consist of a heart valve.

The invention also comprises a through-transmission acoustic system for the repeatable, consistent interrogation of a biological sample arrangement of tissue over a period of time, comprising: at least two transducers arranged in a circuit controlled by a central processing unit; an oscillator arranged in the circuit, to generate a pulse of specific electrical waveforms; an amplifier arranged in the circuit to increase the strength of the pulse of one of the transducers into acoustic energy; an arrangement of electrodes attached to the transducer arrangement, to conduct the pulse into an acoustic wave directed to the sample arrangement; a second of the transducers is arranged to receive the acoustic wave from the one of the transducers and convert the wave into an electrical waveform; a second amplifier in the circuit arranged to condition the wave into time and frequency domains; and a processor for display of spectral components of a signal received by the second transducer over a period of time to present quantitative information relative to changes in the sample arrangement over the time period of interrogation, the sample arrangement comprising a plurality of individual tissue samples disposed among a plurality of multi-well plates. The multi-well plates may be comprised of polystyrene plastic having a flat bottom and a low evaporation lid thereon.

The invention also comprises a tissue implantable pulse-echo acoustic system for the repeatable, consistent interrogation and rf interrogation-reporting relative to an in-vivo or in-vitro biological sample of tissue, over a period of time, comprising: a tissue-implantable transducer arrangement arranged in rf communication with a circuit controlled by a central processing unit; an oscillator arranged in the circuit, to generate a pulse of specific electrical waveforms; an amplifier arranged in the circuit to increase the strength of the pulse of one of the transducers into acoustic energy; an electrode attached to the transducer arrangement, to conduct the pulse into an acoustic wave directed to the sample; a switch arranged to control and re-direct an echo of the acoustic wave from the transducer arrangement and convert the echo into an electrical waveform; a receive amplifier in the circuit arranged to condition the wave into time and frequency domains; and a processor for display of spectral components of a received rf signal redirected by the switch over a predetermined and extended period of time to present quantitative information relative to the sample changes over that time period of interrogation. The invention may include a trocar delivery device for implantation of the transducer arrangement into a living being. The invention includes an obturator which is insertable into the trocar for manipulative introduction into a living being. The transducer arrangement may be disposed within an acoustically-transmissive capsule having a dimensionally-controlled path from the transducer arrangement into the tissue sample being interrogated. The capsule preferably has a dipole antenna therein for transmission and receipt of rf signals over short distances, relative to the transducer arrangement therewithin.

The invention also comprises a method of utilizing the tissue-implantable pulse-echo acoustic system, including one or more of the following preferred embodiments: introducing the capsule into blood vessels of a living being; introducing the capsule into the lymphatic system of a living being; introducing the capsule into the esophagus of a living being; introducing the capsule into the intestine of a living being; introducing the capsule into the intestine of a living being; introducing the capsule into the muscle tissue of a living being; introducing the capsule into the interstitial fluid of a living being;

The invention also comprises an acoustic system for the repeatable, consistent interrogation of a biological sample arrangement of tissue over a period of time, comprising: a contact transducer arranged in acoustical communication with an acoustically transmissive chamber, the chamber containing a self-leveling sample for interrogation; an acoustic matching layer on the transducer, the layer comprised of material of known acoustic impedance as an electrical contact therefor, for facilitating consistent acoustic communication with the sample. The acoustically transmissive chamber may include an array of sample-receiving wells thereon to permit multiple cell line samples to be interrogated simultaneously over a pre-determined period of time. The chamber may be arranged within a controlled environment, wherein a temperature and humidity control mechanism monitors and controls the environment of the chamber.

The invention also comprises a method of acoustically interrogating a plurality of biological samples over an extended predetermined period of time, comprising one or more of the following steps which include: providing a multi-welled plate with a corresponding number of acoustic transducers and a proper circuit in respective acoustic communication therewith; introducing a specific culture medium respectively into a plurality of wells of the multi-welled plate; placing the multi-welled plate with the transducers thereon into an environmentally controlled incubator; applying acoustic energy to the acoustic transducers; growing the culture medium in the wells over the pre-determined period of time; and recording data returned from the transducers over the period of time; interchanging the plates according to proper sampling protocol. The time period may comprise a range of from about two hours to at least forty-eight hours. The method preferably includes reading the results of such sample interrogation over an extended period of time.

The invention also comprises an acoustically transmissive enclosed chamber for sampling a biological sample through a transducer arrangement in communication therewith, wherein the chamber has precise known dimensions and a sample therein has a precise and time-wisely consistent thickness, the sample having a liquid and air interface thereon, wherein the interface comprises an acoustic reflector for quantifying reflected acoustic waves from the transducer arrangement over a predetermined period of time.

The invention also comprises a method of acoustically interrogating a biological sample over a period of time in an acoustically transmissive environmentally enclosed chamber, comprising one or more of the following steps of: measuring the dimensions of acoustically transmissive portions of the chamber for factoring with return echo pulses of the sample in the chamber; providing an acoustic transducer in acoustic communication with the chamber; providing precise quantities of the sample into the chamber; energizing the transducer; sending acoustic waves onto an interface between an upper surface of the sample and any atmosphere within the chamber; and analyzing the acoustic waves received from the interface and received by the transducer arrangement over the period of time. The duration of the period of time may preferably range from at least about two hours to at least about forty-eight hours.

The invention also comprises a method of acoustically interrogating biological samples over a prolonged period, comprising: arranging a series of well plates with biological samples therein, arranging the series of well plates on a corresponding array of acoustic transducers, and acoustically interrogating the biological samples through the well plates over a period of time. The period of time may preferably comprise a range of about two hours to about at least forty-eight hours. The well plates and the samples preferably have precise dimensional characteristics factored into the analysis of interrogation of a sample within the well plates to facilitate cell data comparison and cell characteristics. The well plate may be arranged within an environmentally controlled incubator. The steps of the invention may include measuring and analyzing acoustic wave transmission with respect to a sample/environment interface within the environmentally controlled chamber over an extended period of time.

### BRIEF DESCRIPTION OF THE DRAWINGS

In the drawings, like reference characters generally refer to the same parts throughout the different views. The drawings are not necessarily to scale, emphasis being placed upon illustrating the principles of the invention. The invention will be better understood and objects other than those set forth above will become apparent when consideration is given to the following detailed description thereof. Such description makes reference to and becomes comprised of the annexed drawings wherein:

FIG. 1 is a schematic diagram of a through-transmission acoustic system having at least two transducers and is controlled by a Central Processing Unit (CPU);

FIG. 2 is schematic diagram of a pulse-echo acoustic system having at least one transducer and is controlled by a CPU;

FIG. 3 is a graph of a continuous wave (CW) waveform as typically generated with a through-transmission acoustic system, as displayed in time domain;

FIG. 4 is a graph of an impulse waveform as typically generated in a pulse echo acoustic system, as displayed in the time domain;

FIG. 5 is a partial cross section view of a chamber and an acoustic transducer in contact relationship connected to an acoustic system;

FIG. 6 is a diagrammatic view of a system employing acoustic transducers in contact with a chamber and located in a controlled environment, here, multiple, interchangeable chambers labeled “a” through “e” are shown;

FIG. 7 is a cross-sectional view of an acoustically transmissive chamber comprised of a polymeric material and having specific thickness dimensions through which sound may propagate over time and distance, wherein the chamber may hold at least one human cell and may contain growth medium;

FIG. 8 is a cross section view of a medical device known as a trocar equipped with a delivery system to implant a transducer capsule in a living being;

FIG. 9 is a diagrammatic view of the human breast with a trocar inserted therein;

FIG. 9a is a diagrammatic view of the human breast with an implantable transducer capsule implanted therein; and

FIG. 10 is a cross-sectional view of an interrogation capsule with a dimensionally controlled signal path.

### DETAILED DESCRIPTION OF THE DRAWINGS AND INVENTION

The field of acoustic imaging and sensing typically employs the use of one or more acoustic transducers capable of sending or receiving acoustic energy directed at materials and objects. In a medical application, there is often a region of interest that is defined as an organ or a portion of an organ to which the acoustic energy may be directed and from which it may be received. In typical ultrasound systems, in which the frequency of the acoustic wave is determined to be above the range of human hearing, a wave is generated which may be in the form of a short pulse, or a train of pulses, or a continuous wave (CW) may be generated. Because there is a propagation time that varies in different materials, having different densities, loss mechanisms and scattering, it is possible to examine the wave as it passes through or is otherwise modified by the objects to determine the acoustic properties of these materials. There are two major system configurations used for acoustic interrogation of tissue and biologic samples.

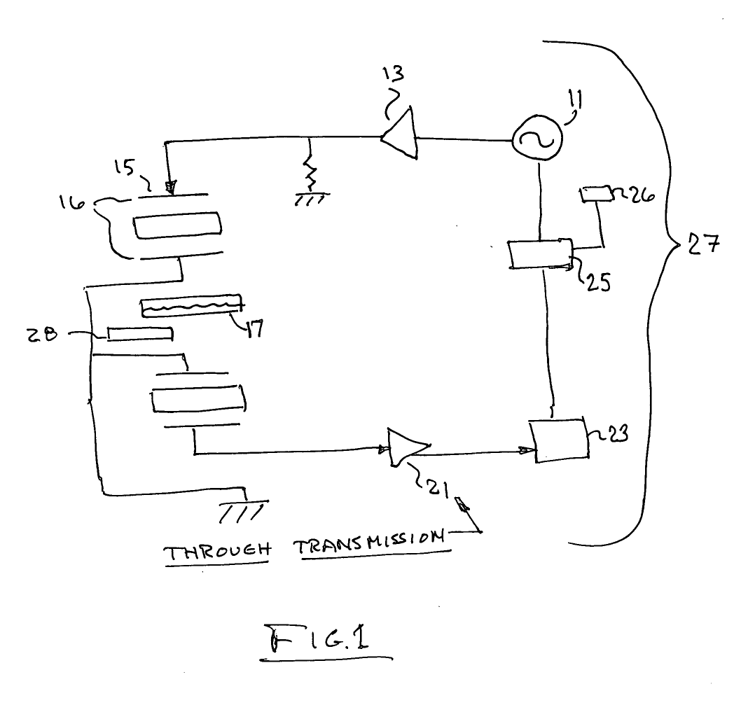

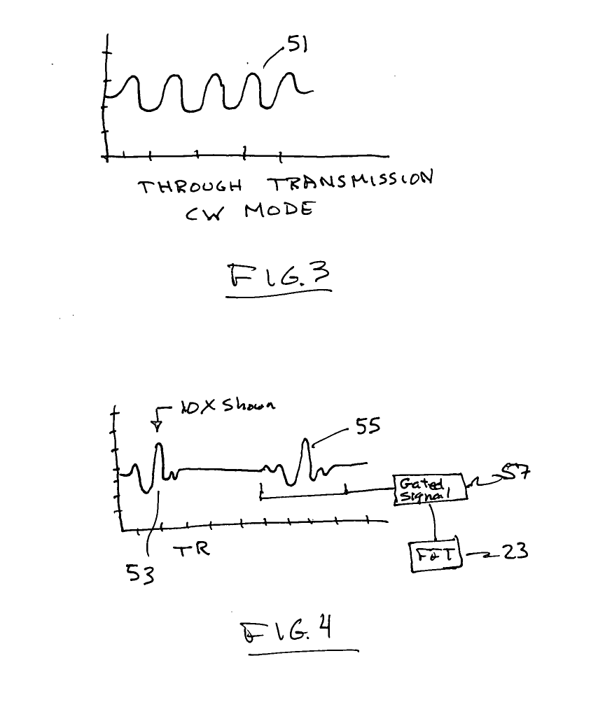

Referring now to FIG. 1, there is shown a through transmission acoustic system or circuit 27 having at least two transducers that are controlled by a central processing unit (CPU) 25. An oscillator 11 serves to generate a pulse or train of pulses or specific electrical waveforms. One frequently used waveform in a through-transmission system is a sinusoidal waveform, represented in FIG. 3, as the graph of a continuous wave CW waveform, for example, a sine wave 51, shown here as a long series of identical waves. The electrical signal may be very short, in the order of GHz, or longer, as found in the audio spectrum.

Referring back once again to FIG. 1, a first amplifier 13 may be used to increase the strength of, and condition the signal in such a way so that a first transducer 15 may efficiently convert the electrical energy into acoustic energy. An electrical impedance matching circuit (not shown) may be used to transform the electrical impedance of the amplifier 13 to the electrical impedance of the first transducer 15 so that efficient and reflection free transmission of energy is allowed to occur. The first transducer 15 may be comprised of for several examples, a piezoelectric material, such as lithium niobate, lead-zirconate-titanate, or Rochelle salt, and various polymeric materials such as polyvinylidene fluoride, otherwise known as PVDF, which may be formed into a membrane or film. When contacted with an arrangement of electrodes 16, and energized, the first transducer 15 may expand and contract, thus generating an acoustic, mechanical wave, which may be directed to an object 17. The object 17 may be any material but in this invention is generally preferably either a biological specimen, such as a breast or an earlobe, a container for a biological specimen, or both. Sound waves emanating from the first transducer 15 may travel through the object 17 and then received by a second transducer 19, which converts the acoustic, mechanical energy into an electrical waveform. Thus, the acoustic signal in a through-transmission system 27 is responsive to changes that may occur during the propagation of that acoustic signal through the object 17, which, when converted back into electrical signal with the second transducer 19, may be then conditioned by a second amplifier 21. Because the spectral, frequency components of the signal are often of most interest, the electrical signal, which is amplified in the time and frequency domains, is detected and analyzed in the frequency domain via software or hardware that performs a Fourier Transform of that signal according to Fourier's Series of Trigonometric Equations. Such a device or system is often referred to as a Fast Fourier Transform or FFT 23, which may output to a central processing unit (CPU) 25 such as a computer and with a display system 26 which depicts quantitative information about the spectral components of the signal received by the second transducer 19. It should be pointed out that the use of the first transducer 15 and the second transducer 19 is merely a convenient arrangement for the through-transmission analysis of material and that other arrangements, such as an array of multiple transducers, or a single transducer arranged with an acoustic mirror 28, may be configured to provide a similar function.

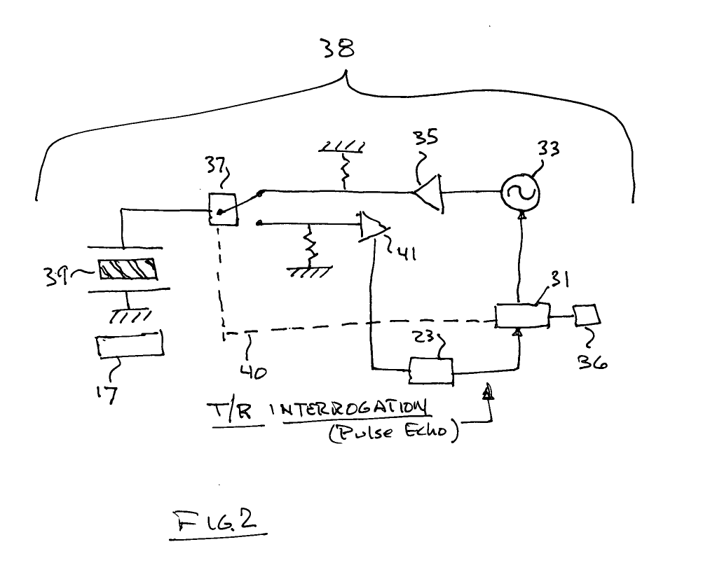

Referring now to FIG. 2, the schematic diagram of a pulse-echo acoustic system having at least one transducer, there is depicted a transmit/receive type acoustic system 38 that is different embodiment in operation than that shown in FIG. 1. Referring once again to FIG. 2, a CPU 31 initiates timing of a pulse generator such as a voltage controlled oscillator 33. The pulse generator may also consist of a high voltage source triggered by a fast field effect transistor (not shown) and passed through amplifier 35 which may condition the signal. A transmit/receive switch 37 applies the signal to the single transducer 39 which is placed in near proximity to the object 17 and subjected to acoustic energy with the single transducer 39. A CPU control line 40 may then rapidly actuate the switch 37 so that the receive amplifier 41 may be properly connected to the single transducer 39 which acts as a converter of acoustic energy into electrical energy that has propagated back via reflection, scattering or other mode from the object 17. A FFT 23 may convert received pulse energy to a spectral format which may then be displayed by a computer 36.

In FIG. 4, the graph of an impulse waveform as typically generated in a pulse echo acoustic system, elucidates the nature of a first “short” pulse 53, which is generated by the system and then a second return pulse 55. The return pulse 55 is received at a later time, as depicted in this time-domain graph. A signal gate 57 times and selects only the reflected signal and directs it in order to exclude energy from the first short pulse from the circuit 53 but allows transmission of the second return pulse 55 to the FFT 23 which analyzes the frequency components of the signal.

An understanding of through-transmission and pulse-echo systems as described in FIG. 1 and FIG. 2, and explained further in FIG. 3 and FIG. 4, will now be essential to comprehension of the features and benefits of the invention as described previously herein and in more detail as follows.

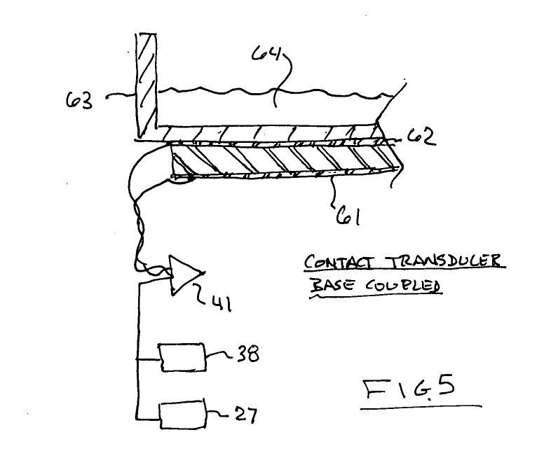

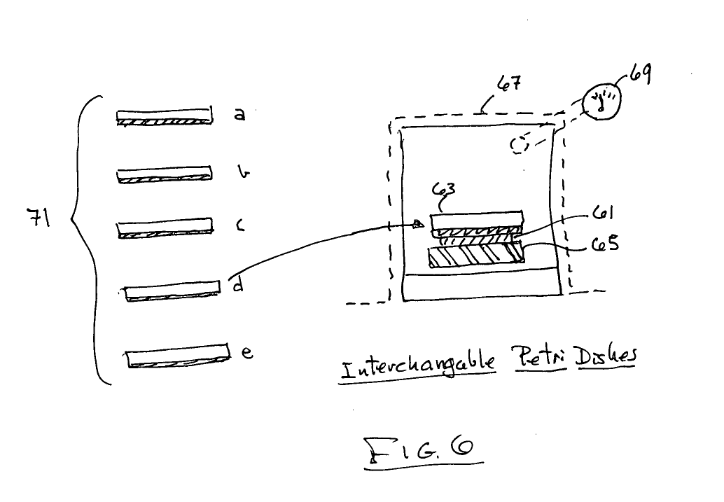

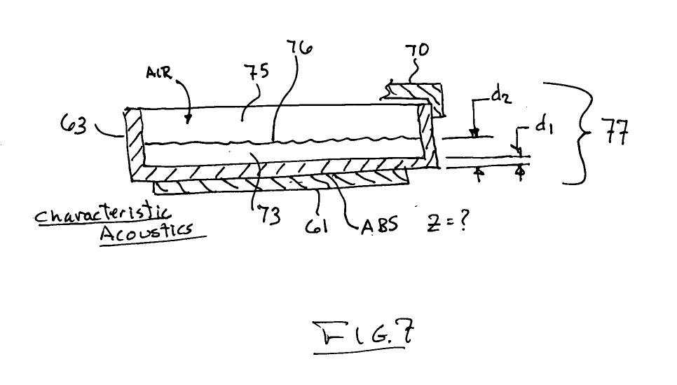

Referring now to FIG. 5, a partial cross-sectional view of a chamber and an acoustic transducer 60 is shown in contact relationship connected to an acoustic system here labeled 38 and 27, corresponding to one of through transmission or pulse echo systems, or both. There is shown in FIG. 5, a contact-transducer 61 in contact with an acoustically transmissive chamber 63 containing a material 64. The contact transducer 61 may be equipped with acoustic an arrangement of matching layers 62 that are comprised of materials of known acoustic impedance that may also serve as an electrical contact, such as for example, a silver-filled epoxy. The material 64 may be a liquid which is self-leveled by gravity, such as water, or a substrate material that may contain cells or other biological components. Registration of the contact transducer 61 in close fitting relationship to the chamber 63 assures that consistent acoustic communication with the material 64 is effected. The chamber 63 may be most conveniently available laboratory ware such as polymeric Petri dishes or disposable, sterile, multi-well plates used commonly for biological experiments especially those in which certain type of cells are grown and cultured in a medium, such as DMEM, or on agarose. The convenience and repeatability, and sterility of the multi-well plates assures that cell lines are not contaminated and allows for many cell line samples or other biological samples to be prepared, and studied with the acoustic apparatus described herein. A preferred multi-well plate may foe example, be an individually packaged Becton Dickenson Multiwell TM 6 well plate number 353046 treated with gas plasma to sterilize it, and which is comprised of polystyrene plastic, and having a flat bottom and a low evaporation lid. Polystyrene is highly transmissive to acoustic energy in the sonic and ultrasonic ranges, exhibits low scattering and viscoelastic loss, and has known and repeatable attenuation as a function of wavelength characteristics, and modest cost which makes it particularly suitable for the purpose of the system which is one object of the present invention. Other materials of well plates that are of various sizes, types, numbers of wells and configurations may be employed as long as they are sterile, repeatable in dimension, and have adequate acoustic transmission properties. Referring now to FIG. 6, the diagrammatic view of a system employing acoustic transducers in contact with a chamber and located in a controlled environment, the advantages of using multi-well disposable sterile chambers become apparent. Various multi-well chambers 71, labeled a-e, may be prepared in advance according to laboratory protocols for sterility, sample handling, titration, and traceability requirements. A laboratory incubator 67 and an arrangement of temperature and humidity controls 69 enclose a transducer base 65, the transducer array 61 and the multi-well plate array 63. The transducer array 65 may contain as many transducers as there are chambers in the well plates and may be arranged in a geometric configuration, such as a rectangular configuration, to correspond with the positions of the similarly configured well plate array 63. Such an arrangement provides a convenient, fast and repeatable method allowing the following: An array plate is prepared with a culture medium, each well is inoculated with a specific cell, and the array plate is placed in the incubator. The acoustic system and transducers apply acoustic energy to the array and the growing cells over a period of time, and data is recorded including frequency domain over time data. The array plates may be interchanged at will according to the protocol being followed in the experiment plan. The array plates may preferably be left in place for a period of about 2 hours to greater than 48 hours and the results read over time.

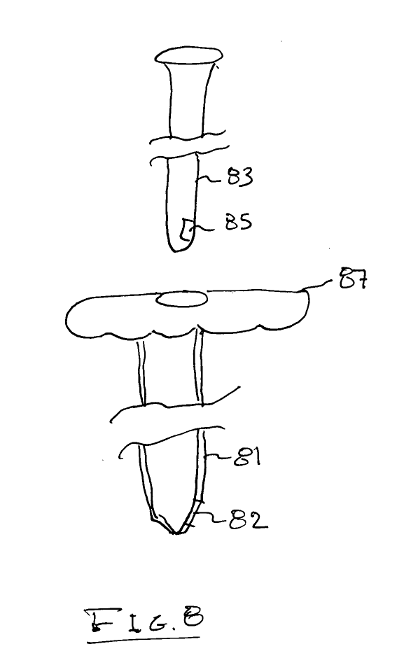

In FIG. 7, a cross-sectional view of an acoustically transmissive chamber is shown, comprised of a polymeric materials having specific thickness dimensions 77, exemplifying the precision and relationship of a fixed acoustic reference and a material to be examined with acoustic waves. The chamber 63 and the fluid 73 may be seen to have parallel, fixed dimensions labeled dSub1 and dSub2 in cross section. Since the acoustic “time of flight” of the chamber material is known from its material and thickness characteristics, the propagation time of an acoustic wave from and to the transducer 61 may be accurately predicted. The culture medium 73, may be applied in precise quantities, will not evaporate easily owing to the temperature and humidity controls used and the use of a low evaporation lid 70. The liquid and air interface 76 distance is also precisely known and controllable and is useful for operating in the through-transmission mode using only a single transducer positioned under the chamber 63. The liquid and air interface acts as an acoustic reflector, redirecting nearly all of the acoustic energy that travels up through the chamber 63 and the fluid 73 back down through the transducer 61. Therefore, it can be seen that that propagation of acoustic energy is governed by lengths dSub1+dSub2+dSub2+dSub1 and held in a specific fixed relationship regardless of small inconsistencies and variations that may occur over the area of the chamber 63 or in the level of the liquid 73. The ability to maintain these relationships over reasonably long periods of time, such as about 2 hours to greater than 48 hours, may be appreciated by practitioners of laboratory biological investigation who required consistency of protocol and maintenance of the operating conditions in a controlled, repeatable manner. Such a condition is a prerequisite for process validation, process control, and the acquisition of valid data sets.

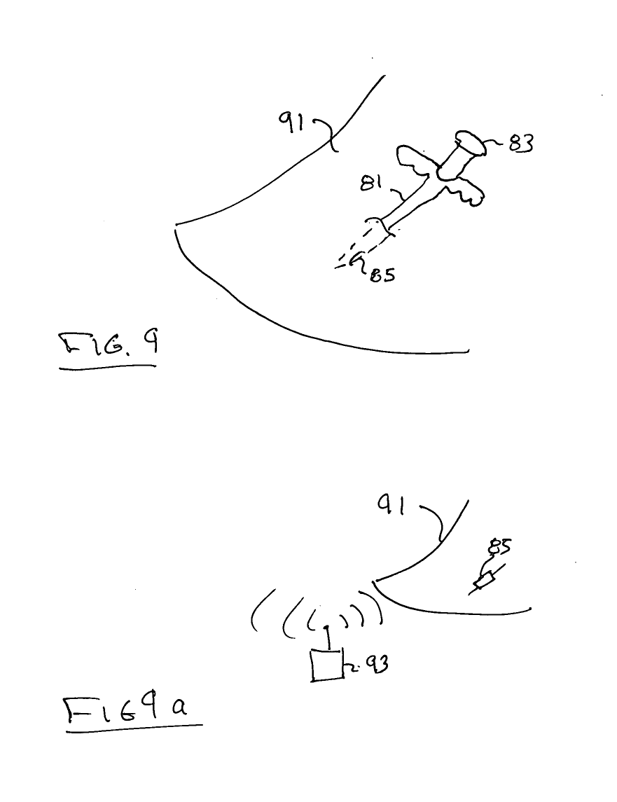

The apparatus and method described thus far provides a more consistent, repeatable and practical acoustic interrogation of biological materials over time than has previously been available. The advantages of various aspects of this apparatus and method once validated through the rigorous protocols thus afforded may now be applied to medical devices which may perform precise analyses of acoustic properties of tissue. Such a medical device may be seen in FIG. 8, a cross sectional view of a medical device known as a trocar equipped with a delivery system to implant a transducer capsule in a living being. The trocar 81 is a hollow, sharp-pointed instrument with an aperture 82 cut into the side or a portion of the wall of the trocar 81. An obturator 83 may be inserted into the hollow end of the trocar 81 so that an acoustic capsule 85 properly registers with the aperture 82.

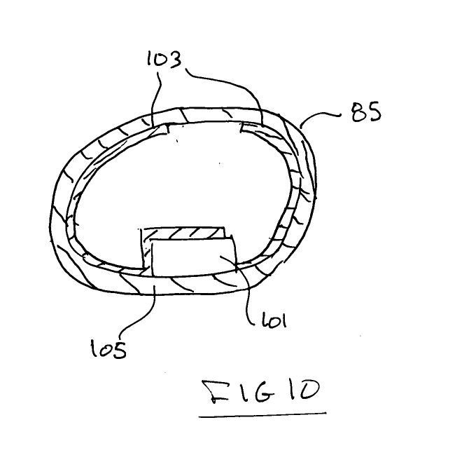

Referring now to FIG. 9, a diagrammatic view of the human breast is shown with a trocar 81 inserted therein. The trocar 81 is inserted directly into a breast 91 with the obturator 83 in place. Manipulation of the obturator 83 releases the capsule 85 which remains in the breast tissue, and then the trocar 81 and the obturator 83 are removed together.

With reference now to FIG. 9a, the diagrammatic view of a human breast with an implanted transducer capsule 85 shown implanted therein. The breast 91 remains intact with the small capsule 85 implanted, which capsule may remain in place for a prolonged period. A radio transceiver 93 is represented communicating with the capsule 85 to interrogate the acoustic properties and other properties of the tissue in contact with the capsule 85.

In reference now to FIG. 10, such an rf transmissive capsule 85 may be fabricated from an acoustically transmissive and dimensionally controlled material similar to that used in the chamber 63 represented in FIG. 7. Returning once again to FIG. 10, the capsule 85 contains a transducer element 101, which may be any of the piezoelectric or other transducer materials as described previously such as for example, PZT. An array of antenna elements 103 are electrically connected to the transducer element 101 at either side, and form a dipole antenna arrangement suitable for transmission and reception over short distances. The capsule wall 105 seals the internal components completely and provides an acoustically transmissive, dimensionally controlled path from the transducer element 101 into the tissue, such as the breast as shown in FIGS. 9 and 9a, and also in other areas of the body such as the prostate, the liver, the various vascular pathways such as the blood vessels, the lymphatic system, the esophagus, the intestine, and in muscle tissue, or in interstitial fluid. The size of the capsule 85 may be quite small, generally down to the relative size of tissue samples being interrogated. Operable acoustically equipped capsules may be fabricated in the range of 4 mm down to less than 0.5 mm using the materials described in the invention.

Thus, what is described is a practical, repeatable acoustic interrogation system and method of operation that is adaptable to laboratory investigations requiring accuracy and rigorous control over experimental conditions and allows rapid, repeatable and large-scale experimentation to proceed in an efficient and cost effective manner. The invention described allows the acoustic interrogation of biological materials over longer time frames with a repeatability and accuracy thus far not available and is therefore capable of monitoring the growth, multiplication, division and death of cells over a prolonged period. The precision and practicality of the apparatus and method may be usefully adapted to disease detecting or monitoring devices and may be implanted in human tissue and read remotely. It should be understood that the foregoing relates to preferred embodiments of the invention and that modifications or alterations may be made without departing from the spirit and scope of the invention as set forth in the description and the appended claims.

I claim:

1. A biological sample acoustic interrogation system having at least one acoustic transducer for interrogating said biological sample, said system comprising:
an acoustic transducer arrangement positioned relative to an acoustically transmissive support, said support arranged in acoustically transmissive contact with said biological sample, said acoustic transducer arrangement arranged in timed acoustic interrogating contact with said support, said timed acoustic interrogating contact comprising a period of about 2 hours to greater than about 48 hours.

2. The biological sample acoustic interrogation system as recited in claim 1, wherein said acoustic transducer arrangement comprises a plurality of individual transducers.

3. The biological sample acoustic interrogation system as recited in claim 1, wherein said acoustic transducer arrangement is fixedly arranged with respect to said acoustically transmissive support.

4. The biological sample acoustic interrogation system as recited in claim 1, wherein said acoustic transducer arrangement is movably arranged with respect to said acoustically transmissive support.

5. The biological sample acoustic interrogation system as recited in claim 1, wherein said acoustically transmissive support is movable arranged with respect to said acoustic transducer arrangement.

6. The biological sample acoustic interrogation system as recited in claim 1, wherein said biological sample comprises at least one biological cell.

7. The biological sample acoustic interrogation system as recited in claim 1, wherein said acoustically transmissive material is a polymeric chamber.

8. The biological sample acoustic interrogation system of claim 7 wherein said polymeric chamber is comprised of a multi-well plate.

9. A method of acoustically interrogating biological samples over an extended time period comprising:
arranging a biological sample for interrogation;
placing said sample in acoustical communication with an acoustic transducer arrangement;
energizing said transducer so as to provide a return signal during an initial time-interrogation event;
energizing said transducer again so as to provide a further return signal during a later time-interrogation event; and
characterizing differences in spectral patterns generated during said initial time-interrogation event and said later time-interrogation event so as to determine sample type and morphology.

10. The method as recited in claim 9, wherein said initial time-interrogation event and said later time-interrogation event are separated by at least 48 hours.

11. The method as recited in claim 9, wherein said acoustic transducer arrangement comprises a pair of spaced-apart acoustic transducers.

12. The method as recited in claim 11, wherein said spaced-apart transducers have said sample arranged therebetween.

13. The method as recited in claim 10, wherein said transducer arrangement is disposed within an implantable medical device.

14. The method as recited in claim 13, wherein said medical device is a stent.

15. The method as recited in claim 13, wherein said medical device is a tissue filter.

16. The method as recited in claim 13, wherein said medical device is an artificial joint.

17. The method as recited in claim 13, wherein said medical device is a heart valve.

18. The method as recited in claim 9, wherein said acoustic transducer arrangement comprises a pulse-echo transducer.

19. The method as recited in claim 9, wherein said acoustic transducer arrangement comprises a through transmission transducer.

20. The method as recited in claim 9, wherein said transducer arrangement is sized to correspond dimensionally to a sample size.

21. The method as recited in claim 9, including:
placing said sample in an acoustically transmissive chamber.

22. The method as recited in claim 21, wherein said acoustically transmissive chamber is comprised of styrene.

23. The method as recited in claim 21, wherein said chamber is comprises of a sterile, non-pyrogenic multiwell plate.

24. The method as recited in claim 21, wherein said chamber has a low evaporation Lid arranged thereon.

25. The method as recited in claim 21, wherein said chamber and said transducer arrangement are disposed in a fixed relationship with one another.

26. The method as recited in claim 21, including:
placing said chamber in a controlled environment prior to energizing of said transducer.

27. The method as recited in claim 9, including:
placing said transducer arrangement in an implantable device.

28. The method as recited in claim 27, including:
placing said implantable device in a living body part.

29. A through-transmission acoustic system for the repeatable, consistent interrogation of a biological sample of tissue over a period of time, comprising:
at least two transducers arranged in a circuit controlled by a central processing unit;
an oscillator arranged in said circuit, to generate a pulse of specific electrical waveforms;
an amplifier arranged in said circuit to increase the strength of said pulse of one of said transducers into acoustic energy;
an arrangement of electrodes attached to said transducer arrangement, to conduct said pulse into an acoustic wave directed to said sample;
a second of said transducers arranged to receive said acoustic wave from said one of said transducers and convert said wave into an electrical waveform;
a second amplifier in said circuit arranged to condition said wave into time and frequency domains; and
a processor for display of spectral components of a signal received by said second transducer over a period of time to present quantitative information relative to said sample changes over said time period of interrogation.

30. The through-transmission system as recited in claim 29, wherein said time period of interrogation of said biological sample of tissue comprises a range of at least two hours up to at least about 48 hours.

31. The through-transmission system as recited in claim 30, wherein said at least two transducers are disposed in a tissue sample-contacting chamber.

32. The through-transmission system as recited in claim 31, wherein said tissue sample-contacting chamber is arranged in vivo.

33. The through-transmission system as recited in claim 32, wherein said chamber is an in vivo medical device.

34. The through-transmission system as recited in claim 33, wherein said medical device consists of a stent.

34. The through-transmission system as recited in claim 33, wherein said medical device consists of a catheter.

35. The through-transmission system as recited in claim 33, wherein said medical device consists of a filter.

36. The through-transmission system as recited in claim 33, wherein said medical device consists of a trocar.

37. The through-transmission system as recited in claim 33, wherein said medical device consists of an artificial joint.

38. The through-transmission system as recited in claim 33, wherein said medical device consists of a needle.

39. The through-transmission system as recited in claim 33, wherein said medical device consists of a free-floating body conveyable through a vascular system.

40. The through-transmission system as recited in claim 33, wherein said medical device consists of a heart valve.

41. A pulse-echo acoustic system for the repeatable, consistent interrogation of a biological sample of tissue over a period of time, comprising:
a transducer arrangement disposed in a circuit controlled by a central processing unit;
an oscillator arranged in said circuit, to generate a pulse of specific electrical waveforms;
an amplifier arranged in said circuit to increase the strength of said pulse of one of said transducers into acoustic energy;
an electrode attached to said transducer arrangement, to conduct said pulse into an acoustic wave directed to said sample;
a switch arranged to control and re-direct an echo of said acoustic wave from said transducer arrangement and convert said echo into an electrical waveform;
a receive amplifier in said circuit arranged to condition said wave into time and frequency domains; and
a processor for display of spectral components of a signal redirected by said switch over a period of time to present quantitative information relative to said sample changes over said time period of interrogation.

42. The through-transmission system as recited in claim 41, wherein said time period of interrogation of said biological sample of tissue comprises a range of at least two hours up to at least about 48 hours.

43. The through-transmission system as recited in claim 41, wherein said transducer arrangement is disposed in a tissue sample-contacting chamber.

44. The through-transmission system as recited in claim 43, wherein said tissue sample-contacting chamber is arranged in vivo.

45. The through-transmission system as recited in claim 44, wherein said chamber is an in vivo medical device.

46. The through-transmission system as recited in claim 45, wherein said medical device consists of a stent.

47. The through-transmission system as recited in claim 45, wherein said medical device consists of a catheter.

48. The through-transmission system as recited in claim 45, wherein said medical device consists of a filter.

49. The through-transmission system as recited in claim 45, wherein said medical device consists of a trocar.

50. The through-transmission system as recited in claim 45, wherein said medical device consists of an artificial joint.

51. The through-transmission system as recited in claim 45, wherein said medical device consists of a needle.

52. The through-transmission system as recited in claim 45, wherein said medical device consists of a free-floating body conveyable through a vascular system.

53. The through-transmission system as recited in claim 45, wherein said medical device consists of a heart valve.

54. A through-transmission acoustic system for the repeatable, consistent interrogation of a biological sample arrangement of tissue over a period of time, comprising:
at least two transducers arranged in a circuit controlled by a central processing unit;
an oscillator arranged in said circuit, to generate a pulse of specific electrical waveforms;
an amplifier arranged in said circuit to increase the strength of said pulse of one of said transducers into acoustic energy;
an arrangement of electrodes attached to said transducer arrangement, to conduct said pulse into an acoustic wave directed to said sample arrangement;
a second of said transducers arranged to receive said acoustic wave from said one of said transducers and convert said wave into an electrical waveform;
a second amplifier in said circuit arranged to condition said wave into time and frequency domains; and
a processor for display of spectral components of a signal received by said second transducer over a period of time to present quantitative information relative to changes in said sample arrangement over said time period of interrogation, said sample arrangement comprising a plurality of individual tissue samples disposed among a plurality of multiwell plates.

55. The through-transmission system as recited in claim 54, wherein said multiwell plates are comprised of polystyrene plastic having a flat bottom and a low evaporation lid thereon.

56. A tissue implantable pulse-echo acoustic system for the repeatable, consistent interrogation and rf interrogation-reporting relative to an in vivo biological sample of tissue, over a period of time, comprising:
a tissue-implantable transducer arrangement arranged in rf communication with a circuit controlled by a central processing unit;
an oscillator arranged in said circuit, to generate a pulse of specific electrical waveforms;
an amplifier arranged in said circuit to increase the strength of said pulse of one of said transducers into acoustic energy;
an electrode attached to said transducer arrangement, to conduct said pulse into an acoustic wave directed to said sample;
a switch arranged to control and re-direct an echo of said acoustic wave from said transducer arrangement and convert said echo into an electrical waveform;
a receive amplifier in said circuit arranged to condition said wave into time and frequency domains; and
a processor for display of spectral components of a received rf signal redirected by said switch over a predetermined period of time to present quantitative information relative to said sample changes over said time period of interrogation.

57. The tissue-implantable pulse-echo acoustic system as recited in claim 56, including a trocar delivery device for implantation of said transducer arrangement into a living being.

58. The tissue-implantable pulse-echo acoustic system as recited in claim 57, including an obturator which is insertable into said trocar for manipulative introduction into a living being.

59. The tissue-implantable pulse-echo acoustic system as recited in claim 56, wherein said transducer arrangement is disposed within an acoustically-transmissive capsule having a dimensionally-controlled path from said transducer arrangement into said tissue sample being interrogated.

60. The tissue-implantable pulse-echo acoustic system as recited in claim 59, wherein said capsule has a dipole antenna therein for transmission and receipt of rf signals over short distances, relative to said transducer arrangement therewithin.

61. A method of utilizing said tissue-implantable pulse-echo acoustic system as recited in claim 59, including:
introducing said capsule into blood vessels of a living being.

62. A method of utilizing said tissue-implantable pulse-echo acoustic system as recited in claim 59, including:
introducing said capsule into the lymphatic system of a living being.

63. A method of utilizing said tissue-implantable pulse-echo acoustic system as recited in claim 59, including:
introducing said capsule into the esophagus of a living being.

64. A method of utilizing said tissue-implantable pulse-echo acoustic system as recited in claim 59, including:
introducing said capsule into the intestine of a living being.

65. A method of utilizing said tissue-implantable pulse-echo acoustic system as recited in claim 59, including:
introducing said capsule into the intestine of a living being.

66. A method of utilizing said tissue-implantable pulse-echo acoustic system as recited in claim 59, including:
introducing said capsule into the muscle tissue of a living being.

67. A method of utilizing said tissue-implantable pulse-echo acoustic system as recited in claim 59, including:
introducing said capsule into the interstitial fluid of a living being.

68. An acoustic system for the repeatable, consistent interrogation of a biological sample arrangement of tissue over a period of time, comprising:
a contact transducer arranged in acoustical communication with an acoustically transmissive chamber, said chamber containing a self-leveling sample for interrogation;
an acoustic matching layer on said transducer, said layer comprised of material of known acoustic impedance as an electrical contact therefor, for facilitating consistent acoustic communication with said sample.

69. The acoustic system as recited in claim 68, wherein said acoustically transmissive chamber includes an array of sample-receiving wells thereon to permit multiple cell line samples to be interrogated simultaneously over a pre-determined period of time.

70. The acoustic system as recited in claim 68, wherein said chamber is arranged within a controlled environment, wherein a temperature and humidity control mechanism monitors and controls said environment of said chamber.

71. A method of acoustically interrogating a plurality of biological samples over an extended predetermined period of time, comprising:
providing a multi-welled plate with a corresponding number of acoustic transducers and proper circuit in respective acoustic communication therewith;
introducing a specific culture medium respectively into a plurality of wells of said multi-welled plate;
placing said multi-welled plate with said transducers thereon into an environmentally controlled incubator;
applying acoustic energy to said acoustic transducers;
growing said culture medium in said wells over said pre-determined period of time; and
recording data returned from said transducers over said period of time.

72. The method as recited in claim 71, including:
interchanging said plates according to proper sampling protocol.

73. The method as recited in claim 71, wherein said time period comprises a range of from about two hours to at least forty-eight hours.

74. The method as recited in claim 71, including:
reading the results of such sample interrogation over time.

75. An acoustically transmissive enclosed chamber for sampling a biological sample through a transducer arrangement in communication therewith, wherein said chamber has precise dimensions and a sample therein has a precise and time-wisely consistent thickness, said sample having a liquid and air interface thereon, wherein said interface comprises an acoustic reflector for quantifying reflected acoustic waves from said transducer arrangement over a predetermined period of time.

76. A method of acoustically interrogating a biological sample over a period of time, in an acoustically transmissive environmentally enclosed chamber, comprising:
measuring the dimensions of acoustically transmissive portions of said chamber for factoring with return echo pulses of said sample in said chamber;
providing an acoustic transducer in acoustic communication with said chamber;
providing precise quantities of said sample into said chamber;
energizing said transducer;
sending acoustic waves onto an interface between an upper surface of said sample and any atmosphere within said chamber; and
analyzing said acoustic waves received from said interface and received by said transducer arrangement over said period of time.

77. The method as recited in claim 76, wherein the duration of said period of time ranges from at least about two hours to at least about forty-eight hours.

78. A method of acoustically interrogating biological samples over a prolonged period, comprising:
arranging a series of well plates with biological samples therein,
arranging said series of well plates on a corresponding array of acoustic transducers, and
acoustically interrogating said biological samples through said well plates over a period of time.

79. The method as recited in claim 78, wherein said period of time comprises a range of about two hours to about at least forty-eight hours.

80. The method as recited in claim 78, wherein said well plates and said samples have precise dimensional characteristics factored into analysis of interrogation of a sample within said well plates.

81. The method as recited in claim 78, wherein said well plate is arranged within an environmentally controlled incubator.

82. The method as recited in claim 81, including:
measuring and analyzing acoustic wave transmission with respect to a sample/environment interface within said environmentally controlled chamber, over an extended period of time.
